(19) United States
(12) Patent Application Publication
(10) Pub. No.: US 2004/0228872 A1
(43) Pub. Date: Nov. 18, 2004

(54) Tyrosine kinase inhibitors as an adjunctive therapy to botulinum toxin treatment

(76) Inventors: Philip Hallinger Bonner, Lexington, KY (US); Roberts Baker, Lexington, KY (US); Shardan Marc Radmanesh, Lexington, KY (US); William Wallace Richardson, Lexington, KY (US)

Correspondence Address: STOCKWELL & ASSOCIATES, PSC, 861 CORPORATE DRIVE, SUITE 201, LEXINGTON, KY, 40503, US

(21) Appl. No.: 10/784,028

(22) Filed: Feb. 21, 2004

Related U.S. Application Data
(60) Provisional application No. 60/449,243, filed on Feb. 21, 2003.

(51) Int. Cl. A61K 39/00; A61K 39/38

(52) U.S. Cl. 424/184.1

(57) ABSTRACT

The present invention is directed to methods for extending the duration of the therapeutic effect of a botulinum toxin in an animal. The methods involve the administration of a botulinum toxin, preferably botulinum toxin A, to treat a condition or disorder, and administration of an inhibitor of the protein tyrosine kinase pathway to an animal, such as a mammal, in particular a human, in an amount sufficient to extend the duration of the therapeutic effect of a botulinum toxin. The inhibitor of the protein tyrosine kinase pathway is preferably genistein or an analogue or prodrug thereof or a pharmaceutically acceptable salt of any of the foregoing.

### CROSS-REFERENCE TO RELATED APPLICATIONS

This application claims the priority of U.S. provisional application Ser. No. 60/449,243, filed Feb. 21, 2003, which is hereby incorporated by reference in its entirety.

### FIELD OF THE INVENTION

The present invention relates to a method of prolonging the effect of botulinum toxin treatment through administering a tyrosine kinase inhibitor as an adjunct.

### BACKGROUND

Botulinum toxin, a toxin produced by Clostridium botulinum, has demonstrated therapeutic value for a broad spectrum of conditions and disorders. The toxin has seven distinct serotypes, designated A-G. Each serotype acts at cholinergic nerves to inhibit the release of acetylcholine, producing local chemical denervation. Consequently, botulinum toxin has been successfully used to treat a number of disorders and conditions which respond to a reduction in the level of activity in the cholinergic nervous system.

The disorders and conditions amenable to botulinum toxin treatment can be roughly divided into those involving the skeletal neuromuscular system and those involving the autonomic nervous system.

The following citations, the disclosures of which are hereby incorporated by reference in their entirety, teach the treatment with botulinum toxin of conditions having skeletal neuromuscular system involvement: U.S. Pat. No. 6,645,496 (tardive dyskinesia), U.S. Pat. No. 6,632,433 (cervical dystonia); U.S. Pat. No. 6,423,319 (muscle injuries); U.S. Pat. Nos. 6,290,961 and 6,319,505 (dystonia), U.S. Pat. No. 5,721,215 (muscle spasms), U.S. Pat. No. 6,620,415 (Parkinson's disease), U.S. Pat. No. 5,183,462 (facial wrinkles. See also Carruthers et al., Therapy with Botulinum Toxin (Jankovic et al. eds.) Ch. 46: 577-595, 1994; Garcia et al., Dermatol Surg Jan;22(1):39-43, 1996 and Brandt et al., Dermatol Surg Nov;24(11):1232-4, 1998 for cosmetic applications), U.S. Pat. No. 5,298,019 (involuntary jaw clenching), U.S. Pat. No. 6,358,917 (downturned mouth), U.S. Pat. No. 6,692,481 (amblyopia. See also Carruthers et al., Can. J. Ophthalmol., vol. 31(7): 389-400, 1996, for ophthalmologic disorders generally).

The following citations, the disclosures of which are hereby incorporated by reference in their entirety, teach the treatment with botulinum toxin of conditions having autonomic nervous system involvement: U.S. Pat. No. 6,683,049 (excessive sweating), U.S. Pat. No. 5,766,605 (excessive salivation, asthma, COPD, excessive stomach acid secretion, spastic colitis and excessive sweating), U.S. Pat. No. 6,649,161 (hypocalcemia), U.S. Pat. No. 6,635,247 (hypoparathyroidism), U.S. Pat. No. 6,585,970 (hypothyroidism), U.S. Pat. No. 6,524,580 (thyroid disorders), U.S. Pat. No. 6,447,785 (hypercalcemia), U.S. Pat. Nos. 6,143,306 and 6,261,572 (pancreatic disorder), U.S. Pat. No. 6,358,513 (Hashimoto's thyroiditis), U.S. Pat. No. 6,416,765 (diabetes), U.S. Pat. No. 6,632,440 (hypersecretion of mucus), U.S. Pat. No. 5,437,291 (disorders of smooth muscles), U.S. Pat. No. 4,932,936 (urinary incontinence), U.S. Pat. No. 6,350,455 (excessive catecholamine secretion) and U.S. Pat. No. 6,328,977 (hyperparathyroidism).

Although the benefits of botulinum toxin treatment are great, the therapeutic effect decreases over time. The duration of the therapeutic result ordinarily lasts from three to six months. After this time the treatment must be repeated. Therefore, it is desirable to develop an adjunctive therapy which increases the duration of the therapeutic effect of botulinum toxin.

### SUMMARY OF INVENTION

According to the present invention, a method of using a tyrosine hydroxylase inhibitor, preferably genistein, as an adjunct to botulinum toxin therapy is disclosed. The use of a tyrosine hydroxylase inhibitor with botulinum toxin therapy increases the duration that botulinum toxin therapy is effective.

### BRIEF DESCRIPTION OF FIGURES

FIG. 1 graphically compares the recovery of the blink response of guinea pigs whose orbicularis oculi muscles were injected with botulinum toxin with or without genistein administration.

FIG. 2 graphically compares the recovery of grip strength in mice whose foreleg flexors were injected with botulinum toxin with or without genistein administration.

### DETAILED DESCRIPTION OF THE INVENTION

A method to prolong the period of clinical response to a therapeutic treatment with botulinum toxin is disclosed. The method involves the administration of an effective amount of a tyrosine kinase inhibitor following botulinum toxin treatment.

### Botulinum Toxin Treatment

All of the botulinum toxin serotypes are known to have clinical utility through the inhibition of cholinergic neurons. The various embodiments of the present invention encompass the administration of at least one serotype or modifications of a serotype or combinations thereof to an animal, such as a mammal, in particular a human.

The present invention does not require a specific mode of treatment with a botulinum toxin. A variety of treatment modes are well known in the art, as disclosed in the citations incorporated by reference above.

The above references are provided as specific embodiments, and the disclosures therein are not meant to limit either the scope of possible methods of administering botulinum toxin, or the scope medical conditions that may fall within the scope of this invention. Other methods of administering botulinum toxin known in the art also fall within the scope of this invention.

### Adjunctive Treatment with a Tyrosine Kinase Inhibitor

The method of the present invention further comprises the administration of an inhibitor of the protein tyrosine kinase pathway in an amount sufficient to prolong the effect of a botulinum toxin treatment. Any inhibitor of the protein tyrosine kinase pathway can be used in the method of the present invention as long as it is safe and efficacious. Herein, “PTK inhibitor” will be used to refer to such compounds and is intended to encompass all compounds that affect the protein tyrosine kinase pathway at any and all points in the pathway.

In one embodiment, the PTK inhibitor is genistein (5,7-dihydroxy-3-(4-hydroxyphenyl)-4H-1-benzopyran-4-one) or a pharmaceutically acceptable, protein tyrosine kinase pathway-inhibiting analogue or prodrug thereof or a pharmaceutically acceptable salt of any of the foregoing. Accordingly, the PTK inhibitor can be a compound of the following formula:

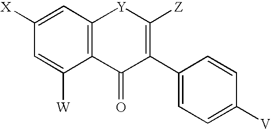

wherein V, W and X are selected from the group consisting of hydro, hydroxyl, alkoxy, halo, an ester, an ether, a carboxylic acid group, a pharmaceutically acceptable salt of a carboxylic acid group, and —SR, in which R is hydrogen or an alkyl group, and Y is selected from the group consisting of oxygen, sulfur, C(OH), and C═O, and Z is selected from the group consisting of hydro and C(O)OR₁, wherein R₁ is an alkyl. Preferably, the alkoxy is a C₁-C₆ alkoxy. Preferably, the halo is fluorine, chlorine or bromine. Preferably, the ester is a C₁-C₆ ester. Preferably, the ether is a C₁-C₆ ether. Preferred pharmaceutically acceptable salts of the carboxylic acid group include sodium and potassium salts. Preferably, the alkyl groups are C₁-C₆ alkyl groups. Desirably, the protein tyrosine kinase pathway inhibitor is genistein.

The prodrug can be any pharmaceutically acceptable prodrug of genistein, a protein tyrosine kinase pathway-inhibiting analogue of genistein, or a pharmaceutically acceptable salt of either of the foregoing. One of ordinary skill in the art will appreciate, however, that the prodrug used must be one that can be converted to an active PTK inhibitor. A preferred prodrug is a prodrug that increases the lipid solubility of genistein, a protein tyrosine kinase pathway-inhibiting analogue of genistein, or a pharmaceutically acceptable salt of either of the foregoing. A preferred prodrug is one in which one or more of V, W and X are independently derivatized with an ester, such as pivalic acid.

Compounds of the above formula are widely available commercially. For example, genistein is available from LC Laboratories (Woburn, Mass.). Those compounds that are not commercially available can be readily prepared using organic synthesis methods known in the art.

Whether a particular analogue, prodrug or pharmaceutically acceptable salt of a compound in accordance with the present invention can prolong the therapeutic effect of botulinum toxin treatment can be determined by its effect in the in vivo model used in Examples 1 and/or 2 below.

The PTK inhibitor can be bound to a suitable matrix, such as a polymeric matrix, if desired, for use in the present inventive method. Any of a wide range of polymers can be used in the context of the present invention provided that, if the polymer-bound compound is to be used in vivo, the polymer is biologically acceptable (see, e.g., U.S. Pat. Nos. 5,384,333 and 5,164,188).

The PTK genistein is very safe and efficacious. For example, genistein is a naturally occurring compound in some foods and populations of Southeast Asians are known to consume on average 70 mg/day of genistein without obvious ill effects.

The PTK inhibitor, which is in the varying embodiments may be genistein, a protein tyrosine kinase pathway-inhibiting analogue of genistein, a protein tyrosine kinase pathway-inhibiting prodrug of genistein, or a pharmaceutically acceptable salt of any of the foregoing, can be administered in accordance with the present inventive method by any suitable route. Suitable routes of administration include systemic, such as orally, by injection or by the implantation of a delivery device such as the VITRASERT® implant manufactured by Bausch & Lomb.

In one embodiment, the PTK inhibitor is administered as soon as possible after botulinum toxin therapy has been performed. Treatment will depend, in part, upon the particular PTK inhibitor used, the amount of the PTK inhibitor administered, the route of administration, and the target or method of botulinum toxin treatment.

One skilled in the art will appreciate that suitable methods of administering a PTK inhibitor, which is useful in the present inventive method, are available. Although more than one route can be used to administer a particular PTK inhibitor, a particular route can provide a more immediate and more effective reaction than another route. Accordingly, the described routes of administration are merely exemplary and are in no way limiting.

The dose administered to an animal, particularly a human, in accordance with the present invention should be sufficient to effect the desired response in the animal over a reasonable time frame. One skilled in the art will recognize that dosage will depend upon a variety of factors, including the strength of the particular PTK inhibitor employed, the age, species, condition or disease state, and body weight of the animal. The size of the dose also will be determined by the route, timing and frequency of administration as well as the existence, nature, and extent of any adverse side effects that might accompany the administration of a particular PTK inhibitor and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. The present inventive method will typically involve the administration of from about 1 mg/kg/day to about 100 mg/kg/day, preferably from about 15 mg/kg/day to about 50 mg/kg/day, if administered systemically.

Compositions for use in the present inventive method preferably comprise a pharmaceutically acceptable carrier and an amount of a PTK inhibitor sufficient to prolong the therapeutic effect of botulinum toxin treatment in vivo. The carrier can be any of those conventionally used and is limited only by chemico-physical considerations, such as solubility and lack of reactivity with the compound, and by the route of administration. It will be appreciated by one of ordinary skill in the art that, in addition to the following described pharmaceutical compositions, the PTK inhibitor can be formulated as polymeric compositions, inclusion complexes, such as cyclodextrin inclusion complexes, liposomes, microspheres, microcapsules and the like (see, e.g., U.S. Pat. Nos. 4,997,652, 5,185,152 and 5,718,922).

The PTK inhibitor can be formulated as a pharmaceutically acceptable acid addition salt. Examples of pharmaceutically acceptable acid addition salts for use in the pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, and arylsulphonic, for example p-toluenesulphonic, acids.

The pharmaceutically acceptable excipients described herein, for example, vehicles, adjuvants, carriers or diluents, are well known to those who are skilled in the art and are readily available to the public. It is preferred that the pharmaceutically acceptable carrier be one which is chemically inert to the PTK inhibitor and one which has no detrimental side effects or toxicity under the conditions of use.

The choice of excipient will be determined in part by the particular PTK inhibitor, as well as by the particular method used to administer the composition. Accordingly, there is a wide variety of suitable formulations of the pharmaceutical composition of the present invention. The following formulations are merely exemplary and are in no way limiting.

Injectable formulations are among the embodiments in accordance with the present inventive method. The requirements for effective pharmaceutical carriers for injectable compositions are well known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well known to those of skill in the art. Such formulations are suitable in the context of the present invention for application to the skin. The use of patches, ophthalmic solutions (see, e.g., U.S. Pat. No. 5,710,182) and ointments, e.g., eye drops, is also within the skill in the art.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inhibitor can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof.

The parenteral formulations will typically contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17.

The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Such compositions can be formulated as sustained-release formulations or devices (see, e.g., U.S. Pat. No. 5,378,475). For example, gelatin, chondroitin sulfate, a polyphosphoester, such as bis-2-hydroxyethyl-terephthalate (BHET), or a polylactic-glycolic acid (in various proportions) can be used to formulate sustained-release formulations. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit (e.g., 100 μm-1 mm in diameter), or an implant or a device comprised of a polymeric composition as described above, can be used.

The present inventive method also can involve the co-administration of other pharmaceutically active compounds. By “co-administration” is meant administration before, concurrently with, e.g., in combination with the PTK inhibitor in the same formulation or in separate formulations, or after administration of a PTK inhibitor as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered. In addition, other types of inhibitors of the protein tyrosine kinase pathway, which include natural protein tyrosine kinase inhibitors like quercetin, lavendustin A, erbstatin and herbimycin A, and synthetic protein tyrosine kinase inhibitors like tyrphostins (e.g., AG490, AG17, AG213 (RG50864), AG18, AG82, AG494, AG825, AG879, AG1112, AG1296, AG1478, AG126, RG13022, RG14620 and AG555), dihydroxy- and dimethoxybenzylidene malononitrile, analogs of lavendustin A (e.g., AG814 and AG957), quinazolines (e.g., AG1478), 4,5-dianilinophthalimides, and thiazolidinediones, can be co-administered with genistein or an analogue, prodrug or pharmaceutically acceptable salt thereof (see Levitzki et al., Science 267: 1782-1788 (1995), and Cunningham et al., Anti-Cancer Drug Design 7: 365-384 (1992)). In this regard, potentially useful derivatives of genistein include those set forth in Mazurek et al., U.S. Pat. No. 5,637,703. Neutralizing proteins to growth factors, such as a monoclonal antibody that is specific for a given growth factor, e.g., VEGF (for an example, see Aiello et al., PNAS USA 92: 10457-10461 (1995)), or phosphotyrosine (Dhar et al., Mol. Pharmacol 37: 519-525 (1990)), can be co-administered. Other various compounds that can be co-administered include inhibitors of protein kinase C (see, e.g., U.S. Pat. Nos. 5,719,175 and 5,710,145), cytokine modulators, an endothelial cell-specific inhibitor of proliferation, e.g., thrombospondins, an endothelial cell-specific inhibitory growth factor, e.g., TNF.alpha., an anti-proliferative peptide, e.g., SPARC and prolferin-like peptides, a glutamate receptor antagonist, aminoguanidine, an angiotensin-converting enzyme inhibitor, e.g., angiotensin II, calcium channel blockers, .psi.-tectorigenin, ST638, somatostatin analogues, e.g., SMS 201-995, monosialoganglioside GM1, ticlopidine, neurotrophic growth factors, methyl-2,5-dihydroxycinnamate, an angiogenesis inhibitor, e.g., recombinant EPO, a sulphonylurea oral hypoglycemic agent, e.g., gliclazide (non-insulin-dependent diabetes), ST638 (Asahi et al., FEBS Letter 309: 10-14 (1992)), thalidomide, nicardipine hydrochloride, aspirin, piceatannol, staurosporine, adriamycin, epiderstatin, (+)-aeroplysinin-1, phenazocine, halomethyl ketones, anti-lipidemic agents, e.g., etofibrate, chlorpromazine and spinghosines, aldose reductase inhibitors, such as tolrestat, SPR-210, sorbinil or oxygen, and retinoic acid and analogues thereof (Burke et al., Drugs of the Future 17(2): 119-131 (1992); and Tomlinson et al., Pharmac. Ther. 54: 151-194 (1992)). Selenoindoles (2-thioindoles) and related disulfide selenides, such as those described in Dobrusin et al., U.S. Pat. No. 5,464,961, are useful protein tyrosine kinase inhibitors. Those patients that exhibit systemic fluid retention, such as that due to cardiovascular or renal disease and severe systemic hypertension, can be additionally treated with diuresis, dialysis, cardiac drugs and antihypertensive agents.

### EXAMPLE 1

This example demonstrates that genistein prolongs the paralytic effects of botulinum toxin on orbicularis oculi muscles.

Surgical procedures. Guinea pigs weighing approximately 500-600 g were anesthetized and small, 4-6 mm slits were cut through the skin and dermis above each of the two eyelids and blunt instruments were used to separate tissues to expose the fasciae of the orbicularis oculi muscles. botulinum toxin A (125 μl, 10 u/ml Botox® or Botox Cosmetic®, Allergan, Inc.) or saline was injected on to and near the muscles. After injection, slow-release implants containing either genistein or vehicle alone were packed into the slits near the muscles, the slits sutured closed, and the animal allowed to recover in an approved facility.

Slow-release implants were discs, 2 mm in diameter by 1 mm thick, manufactured and provided by Control Delivery Systems Inc. of Watertown, Mass. The implants were provided pre-loaded with genistein or vehicle. Genistein containing implants have been shown by the manufacturer to release genistein for at least three months. Inquiries regarding how to obtain or use the implants may be directed to Control Delivery Systems using Reference Number CDS-GI-002.

Various placements of the slow-release implants were tried with best results to occurring when two discs were placed near both the nasal and temporal ends of the orbicularis muscles. Slow-release implants placed in the central portion of the eyelid were also effective, but results were not as consistent as with lateral placements.

Before surgery, and starting again three to four days after surgery, animals were placed in a loose restraint device, calibrated search coils (2 mm in diameter by 1 mm thick, ˜60 turns of 50 AWG copper wire, made in the lab) attached to an eyelid, and blink amplitude measurements collected using MacLab® software (ADInstruments Pty Ltd.). Post-surgical blink data collection was repeated twice a week until the animals were killed and tissues recovered for histological analysis. Blinks were induced by air puffs (5-15 psi) approximately every 10 sec with occasional 60 sec rest periods. Blinks were not induced if the animal's upper eyelid was not at the full open position as judged by whether or not the lid margin was at the level of the limbus. If the lid was not fully open, the animal was allowed to rest until the lid opened fully. Sessions lasted no more than 30 minutes.

### Results

Time to recovery of 50% blink amplitude. Each experimental group was comprised of four guinea pigs. The orbicularis oculi muscles of two groups of guinea pigs were injected with botulinum toxin A. One of these groups also received slow release implants with genistein, while the other group received implants containing vehicle-only (placebo).

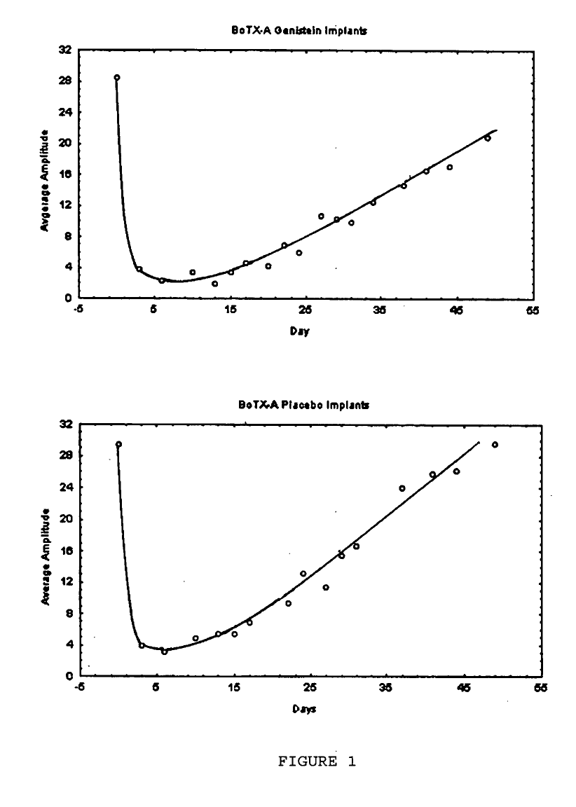

FIG. 1 displays the recovery of blink amplitudes in the groups injected with botulinum toxin A. The points are the means of blink amplitudes determined within a 3-day window. The X-axis is days post-surgery; the Y-axis, mean blink amplitude in degrees. The time to 50% recovery of blinks is delayed approximately 10 days by genistein.

To demonstrate that genistein did not have a deleterious effect on blink amplitude, the orbicularis oculi muscles of two additional groups were injected with saline vehicle instead of botulinum toxin A. One of these groups received slow release implants with genistein, while the other group received implants containing vehicle-only (placebo).

Animals that received saline instead of botulinum toxin A along with genistein implants or placebo implants show only brief, slight impairment of blinks after surgery (˜10% reduction of amplitude at most) and full recovery within a week.

### EXAMPLE 2

This example demonstrates that genistein prolongs the paralytic effects of botulinum toxin on foreleg flexor muscles.

Botulinum toxin type A was injected into the foreleg flexor muscles of two adult mice. At the time of injection, sustained-release implants were placed within the flexor muscles of each mice. One mouse received an implant containing genistein while the other contained vehicle only (placebo). At various times after treatment the strength of the flexor muscles was assayed by measuring the forepaw grip strength. Mice were manipulated so they reflexively grabbed a trapeze with their forepaws. The trapeze was connected to a force transducer. To measure forepaw grip strength they were pulled backwards until they released the trapeze. The force exerted at release is the grip strength.

### Results

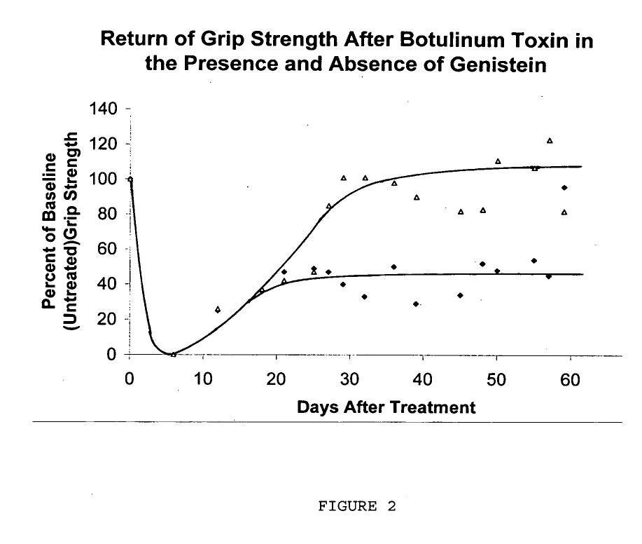

FIG. 2 illustrates the change in grip strengths before and at several times after botulinum toxin treatment. Grip strength is measured in the figure as percent of the untreated baseline grip strength. Grip strength recovery after treatment with botulinum toxin type A is retarded in the presence of genistein (filled diamonds) versus botulinum toxin type A alone (open triangles).

While this invention has been described with an emphasis upon preferred embodiments, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention.

We claim:

1. A method of extending the duration of the therapeutic effect of botulinum toxin in an animal comprising the steps of administering a botulinum toxin to treat a condition or disorder and administering an inhibitor of the protein tyrosine kinase pathway in an amount sufficient to extend the duration of the effect of botulinum toxin therapy.

2. The method of claim 1, wherein said inhibitor of the protein tyrosine kinase pathway is a compound of formula: 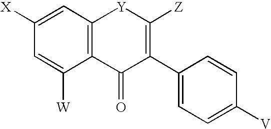
wherein V, W and X are selected from the group consisting of hydro, hydroxyl, alkoxy, halo, an ester, an ether, a carboxylic acid group, a pharmaceutically acceptable salt of a carboxylic acid group, and —SR, in which R is hydrogen or an alkyl group, and Y is selected from the group consisting of oxygen, sulfur, C(OH), and C═O, and Z is selected from the group consisting of hydro and C(O)OR₁, wherein R₁ is an alkyl, or a protein tyrosine kinase-inhibiting prodrug or pharmaceutically acceptable salt thereof.

3. The method of claim 2, wherein said condition or disorder is a condition or disorder involving the skeletal neuromuscular system.

4. The method of claim 2, wherein said condition or disorder is a condition or disorder involving the autonomic nervous system.

5. The method of claim 2, wherein said inhibitor of the protein tyrosine kinase pathway is genistein.

6. The method of claim 5, wherein the dose of genistein administered is from 1 mg/kg/day to about 100 mg/kg/day.

7. The method of claim 5, wherein the dose of genistein administered is from 15 mg/kg/day to about 50 mg/kg/day.

8. The method of claim 5, wherein said botulinum toxin is botulinum toxin type A.

9. The method of claim 5, wherein the botulinum toxin is selected from the group consisting of botulinum toxin types A, B, C, D, E, F and G or a combination thereof.

10. The method of claim 8, wherein said condition or disorder is a condition or disorder involving the skeletal neuromuscular system.

11. The method of claim 8, wherein said condition or disorder is a condition or disorder involving the autonomic nervous system.

12. The method of claim 9, wherein said condition or disorder is a condition or disorder involving the skeletal neuromuscular system.

13. The method of claim 9, wherein said condition or disorder is a condition or disorder involving the autonomic nervous system.

14. The method of claim 1, wherein the botulinum toxin is selected from the group consisting of botulinum toxin types A, B, C, D, E, F and G or a combination thereof.

15. The method of claim 1, wherein the botulinum toxin is botulinum toxin type A.

16. The method of claim 1, wherein said condition or disorder is a condition or disorder involving the skeletal neuromuscular system.

17. The method of claim 1, wherein said condition or disorder is a condition or disorder involving the autonomic nervous system.

18. The method of claim 15, wherein said condition or disorder is a condition or disorder involving the skeletal neuromuscular system.

19. The method of claim 15, wherein said condition or disorder is a condition or disorder involving the autonomic nervous system.

20. The method of claim 14, wherein said condition or disorder is a condition or disorder involving the skeletal neuromuscular system.
